(19) United States
(12) Patent Application Publication
(10) Pub. No.: US 2012/0100547 A1
(43) Pub. Date: Apr. 26, 2012

(54) Real Time PCR Through Gigahertz or Terahertz Spectrometry

(76) Inventor: Tanja Danker, Munchen (DE)

(21) Appl. No.: 13/132,328

(22) PCT Filed: Nov. 30, 2009
(86) PCT No.: PCT/EP09/66081

§ 371 Date: Dec. 21, 2011

(30) Foreign Application Priority Data
Dec. 2, 2008 (DE) 10 2008 059 985.9

(51) Int. Cl. C12Q 1/68; C12M 1/34

(52) U.S. Cl. 435/6.12; 435/287.2

(57) ABSTRACT

A method is provided for spectroscopic real-time detection of nucleic acid molecules, in particular polynucleotide sequences, in a PCR amplification (PCR=polymerase chain reaction). The PCR amplification process includes preparing the initial substances required for producing a PCR solution in a buffer, and the repeating PCR reaction steps of denaturing, primer hybridization and elongation. Electromagnetic radiation is irradiated into the PCR solution during the PCR amplification at defined time points from a radiation source in the gigahertz or terahertz regime in order to detect at least the presence or absence of a polynucleotide sequence using a detector in a real-time detection.

### CROSS-REFERENCE TO RELATED APPLICATIONS

This application is a Section 371 of International Application No. PCT/EP2009/066081, filed Nov. 30, 2009, which was published in the German language on Jun. 10, 2010, under International Publication No. WO 2010/063683 A1 and the disclosure of which is incorporated herein by reference.

### BACKGROUND OF THE INVENTION

The invention relates to a method for the spectroscopic real-time detection of nucleic acid molecules, in particular of polynucleotide sequences, in a PCR amplification process, as well as a corresponding PCR arrangement (PCR=polymerase chain reaction).

Such methods, as well as PCR arrangements, are typically used for the quantitative analysis of the expression of certain gene sequences. PCR amplification processes generally often find use in the amplification and examination of DNA or RNA sequences, as they are found in many biological systems, but can also be used, in principle, with synthetically produced polynucleotide sequences.

The method described in the present case relates to a spectroscopic real-time detection in a PCR amplification process. This is related both to detections during the course of all reaction steps of a PCR amplification process and also to appropriate detections before and after. Thus, for example, substances used in introductory steps before the execution of the actual PCR reaction steps for the replication of polynucleotide sequences can be examined qualitatively, as well as quantitatively, by a spectroscopic detection. Furthermore, spectroscopic examination steps can likewise be performed during the PCR amplification process and also when the PCR amplification process has already been completed.

PCR amplification processes are typically used, with respect to the human genome, to amplify relatively short polynucleotide sequences clearly defined in their composition. In contrast to living organisms, the PCR amplification process is able to guarantee the amplification of only polynucleotide sequences having a few thousand base pairs per sequence. For the execution of a PCR amplification process, it initially requires the production of a PCR solution having, in addition to a buffer solution as a suitable chemical environment, a number of other initial substances necessary for the course of a PCR amplification process. These initial substances typically comprise an original polynucleotide sequence to be amplified, primer for fixing an initial section and also a final section of the polynucleotide sequence to be amplified, in order to replicate the section defined by the primer, as well as deoxynucleoside triphosphates that represent the building blocks of the replicated polynucleotide sequence. In addition to these initial substances, a PCR solution can also include a number of additional functional components that allow, for example, the increase or the optimization of the course of the PCR amplification process with respect to effectiveness.

A PCR amplification process comprises a repeating series of PCR reaction steps, like those that can be performed, for example, in typical laboratory thermocyclers. Each repetition here comprises the three PCR reaction steps of denaturation, primer hybridization, and elongation. Denaturation initially leads to a melting of double-stranded polynucleotide sequences, while heating the PCR solution to a temperature of approximately 95° C., in order to dissolve the hydrogen bridge bonds that hold the two single-stranded polynucleotide sequences together. The temperature level is maintained until it is guaranteed that single-stranded polynucleotide sequences are still present in the PCR solution. In the PCR reaction step of primer hybridization, the temperature is typically held for a few seconds at a level that allows a specific accumulation of the primer on the polynucleotide sequence. The designated temperature level normally lies a few ° C. below the melting point of the primer sequences produced by the specific accumulation and usually corresponds to a temperature between 55° C. and 65° C. If the accumulation of the primer at the predetermined positions of the polynucleotide sequence is stopped, in the PCR reaction step of elongation, the filling by the missing strands or building blocks at free nucleotides under the effect of the polymerase is performed. Here, the primer forms the start of a new single strand, which is replicated piece by piece. The temperature to be set during this PCR reaction step depends on the working optimum of the polymerase to be used, but typically lies 10° C. to 15° C. above the temperature level of the primer hybridization. After completion of the PCR reaction step of elongation, the reaction series repeats and the desired polynucleotide sequences increase exponentially in the best reaction case, because in each additional reaction step, the previously synthesized polynucleotide sequences can also be used as templates for an additional strand synthesis.

In the real-time quantitative PCR, a development of the PCR amplification process known from the prior art, a PCR amplification process is performed for the replication of polynucleotide sequences, and simultaneously a possibility of the quantification of the polynucleotide sequences obtained in this way is provided. The quantification here is based upon the execution of fluorescence measurements, which are performed during the course of the PCR amplification process. The detected fluorescence signal here increases proportionally with the amount of replicated polynucleotide sequences. Accordingly, in contrast to most conventional quantitative detection methods, which can be used only after the PCR amplification process, the real-time quantitative PCR still allows a quantitative evaluation of the polynucleotide sequences replicated in the PCR amplification process during their amplification.

In this respect, it should be noted that here and in the following a quantitative detection is to be understood in the sense of quantification in connection with a real-time quantitative PCR. This can be based on various known quantification models. In a simple case, for example, the fact can be used that a linear, inversely proportional relationship exists between the logarithm of the used quantity of the polynucleotide sequence to be replicated and the CT (Threshold Cycle, i.e., the cycle at which the detected fluorescence initially lies significantly above the background fluorescence). If the quantity used is, for example, initially known, then a standard curve can be generated, against whose increase any unknown quantity of polynucleotide sequence can be compared and quantified. On the other hand, if the starting quantity is unknown, sometimes through the determination of the CT, the starting quantity can also be determined. Additional calculation methods are known to the skilled person in the art.

The real-time quantitative PCR here requires fluorescence dyes or so-called fluorescence markers, which interact chemically or physically with the replicated polynucleotide sequences and consequently change their own fluorescence response. Accordingly, the increase in the intensity of a detected fluorescence signal according to the repeating PCR reaction steps correlates with a corresponding increase in replicated polynucleotide sequences. The detection of the profile of a PCR amplification process by measured fluorescence signals can be supported, for example, with the use of simple fluorescence dyes, which bind in a relatively unspecific way to the polynucleotide sequence to be replicated. Such fluorescence dyes are, for example, SybrGreen, which is an asymmetric cyanine dye molecule and forms a DNA fluorescence dye complex with DNA molecules to be replicated, wherein this complex absorbs blue light at a wavelength of λₘₐₓ=494 nm and emits green light at a wavelength of λₘₐₓ=521 nm. Another frequently used fluorescence dye is ethidium bromide. In addition to these fluorescence dyes, fluorescence markers, such as FRET probes, Lightcycler® probes, TaqMan® probes, molecular beacons, Scorpion primer, or also Lux® primer are also used, which do indeed allow a specific binding at individual points of the polynucleotide sequence and consequently have tightly defined fluorescence properties, but are comparatively expensive. Consequently, such fluorescence markers are not found in wide use as is typical, for example, for fluorescence dyes. Furthermore, for example, molecular beacons represent relatively complex structures that require additionally optimized and thus expensive primers for performing the PCR amplification process. Furthermore, such complex structures make an optimized setting of the PCR reaction conditions more difficult and thus lead to a relatively inefficient amplification of the polynucleotide sequences and also the presentation of many undesired byproducts.

For avoiding these disadvantages of real-time quantitative PCR concerning the detection of fluorescence signals, International patent application publication No. WO 03/102238 A2, known from the prior art, proposes a method for the detection of the presentation of coherent nucleic acid molecules in a PCR solution, as well as an arrangement, in order to perform the method. The method comprises the preparation of PCR initial substances in a chamber, as well as the execution of the repeating PCR reaction steps of denaturation, primer hybridization, and elongation. During the execution of these PCR reaction steps, the presentation of continuous nucleic acid molecules is performed by the irradiation of UV light into the PCR solution and the subsequent detection of the absorbed light intensity. The PCR solution is here absorbed by a chamber that is adapted for the execution of a PCR amplification process and into which the light energy of the UV light source is irradiated.

Although the method described in WO 03/102238 A2 makes the use of fluorescence markers unnecessary, this method presents itself as very susceptible to faults with respect to individual components of the PCR solution or with respect to remaining residue from reaction steps of prior isolation reactions, e.g., phenols. Another disadvantage of the quantitative determination by UV absorption is also to be seen in that single-stranded nucleic acid molecules can be distinguished from double-stranded nucleic acid molecules only to a limited extent, and consequently only a very unspecific detection possibility is available for polynucleotide sequences.

International patent application publication No. WO 2008/109706 A1 describes the use of terahertz spectroscopy in many different ways. In that document, the detection of double-stranded and single-stranded DNA is also described. A real-time method for the detection of the PCR product is described in no way in that publication.

International patent application publication No. WO 2006/064192 A1 describes a band filter in the terahertz range. A method with respect to the detection of nucleic acid molecules is not described in that publication.

U.S. patent application publication No. 2006/0216742 A1 describes a method and a system for the detection of biomolecular bonding events with the use of gigahertz or terahertz radiation.

International patent application publication No. WO 03/100396 A1 discloses a method for the detection of the specificity of a ligand for a biological sample.

German published patent application DE 100 54 476 A1 describes a method for the detection of polynucleotide sequences, in which the index of refraction or an equivalent parameter of the sample in contact with the test medium is determined by interaction with incident electromagnetic radiation.

International patent application publication No. WO 2004/024949 A2 describes a method for the quick detection of mutations and nucleotide polymorphisms with use of spectral data.

A more specific method for the detection of the presence of a biomolecular bond between two molecules is proposed by U.S. 2006/0216742 A1, which relates to a detection method, as well as a corresponding device. According to the disclosure, by terahertz radiation, the presence of a biomolecular bond of two molecules is detected, in that terahertz radiation is emitted from a source and is detected by a detector after passing through a sample comprising one of the molecules. Here, however, the disclosed method does not allow the quantification of quantities of the molecules used and is consequently unsuitable for use for real-time detection in a PCR amplification process.

### BRIEF SUMMARY OF THE INVENTION

Consequently, the object presents itself to provide a marker-free method for the real-time detection of polynucleotide sequences in a PCR amplification process, which allows a specific and quantitative detection of polynucleotide sequences, in particular, a difference between single-stranded and double-stranded polynucleotide sequences and is not essentially influenced by the purity or quality of the solution.

In particular, the object is achieved by a method for both the quantitative spectroscopic real-time detection of nucleic acid molecules, in particular of polynucleotide sequences, in a PCR amplification process, wherein the PCR amplification process comprises the preparation of the initial substances necessary for the production of a PCR solution in a buffer solution, as well as the repeating PCR reaction steps of denaturation, primer hybridization, and elongation, and wherein, during the PCR amplification process, electromagnetic radiation in the gigahertz or terahertz regime is irradiated into the PCR solution from a radiation source at defined time points, in order to detect at least the presence or absence of a polynucleotide sequence by a detector in a quantitative, real-time detection process.

Furthermore, the object is achieved by a method for both the quantitative spectroscopic real-time detection of nucleic acid molecules, in particular of polynucleotide sequences, in a PCR amplification process, wherein the PCR amplification process comprises the preparation of the initial substances necessary for the production of a PCR solution in a buffer solution, as well as the repeating PCR reaction steps of denaturation, primer hybridization, and elongation, and wherein, during the PCR amplification process, electromagnetic radiation in the gigahertz or terahertz regime is irradiated into the PCR solution from a radiation source at defined time points, in order to detect at least the presence or absence of a polynucleotide sequence by a detector in a real-time detection process, and wherein the reaction step of denaturation is caused by terahertz radiation from the radiation source.

Moreover, the object is achieved by a PCR arrangement for both the quantitative spectroscopic real-time detection of polynucleotide sequences in a PCR amplification process, wherein this comprises a substrate with an absorption area for a PCR solution consisting of the necessary initial substances and a buffer solution, temperature-change device by which the PCR solution can be heated and/or cooled to specified temperatures, in order to allow the repeating PCR reactions steps of denaturation, primer hybridization, or elongation, a radiation source, in order to irradiate electromagnetic radiation in the gigahertz or terahertz regime into the PCR solution, and a detector, in order to detect radiation transmitted through the PCR solution also in a quantitative, spectroscopic manner.

A core concept of the present methods, as well as the PCR arrangement according to the invention, lies in the execution of a PCR amplification process, as well as a spectroscopic real-time detection process in the gigahertz or terahertz regime of the electromagnetic spectrum. Radiation typically used in this frequency range lies between 100 gigahertz and 20 terahertz, in particular between 300 gigahertz and 10 terahertz. These radiation frequencies are suitable for exciting certain excited states (typically vibration oscillation modes) of single-stranded and double-stranded polynucleotide sequences. Excited states of individual, functional groups of these polynucleotide sequences are consequently correlated with a predetermined frequency of electromagnetic radiation and can be detected in extinction measurements. Under extinction measurements, below, in general, all spectroscopic detections, likewise quantitative, are designated that set parts of the light intensity irradiated into the PCR solution in relationship with parts of the light intensity discharged from it and detected with a detector, in order to allow conclusions to be made on substances and structures to be detected in the PCR solution. These excitation modes (excitation frequencies) are moreover characteristic for the chemical bonding states of these functional groups, and consequently a change in the bonding state can be accompanied by a change in the electromagnetic excitation frequency. In particular, excitation modes can be detected whose appearance is characteristic for the presence of hydrogen bridge bonds between the two single-stranded polynucleotide sequences in a double-stranded polynucleotide sequence. Accordingly, through extinction measurements in this designated frequency range, quantitative conclusions can be obtained on whether a mixture of single-stranded or double-stranded polynucleotide sequences is present in a PCR solution.

The detection of predetermined excited states of polynucleotide sequences in a PCR solution by spectroscopic extinction measurements requires, in comparison with conventional methods of detection by fluorescence measurements, absolutely no fluorescence markers, which must be added to a PCR solution. With this configuration, on the one hand, the preparation of a PCR solution has a less complex and more economical construction. On the other hand, the use of UV light, which is harmful for the PCR solution can be eliminated. Just by UV light, which causes electronic excitations in biological macromolecules, the possible sources of error by secondary chemical reactions, which are consequently initiated by the irradiation of UV light, are very problematic. Electromagnetic irradiation in the gigahertz as well as terahertz regime, however, typically allow only vibration excitations to be generated and thus rarely disrupt the chemical reaction environment in a PCR solution from a practical viewpoint. Moreover, the radiation frequency of gigahertz and terahertz radiation is suitable to detect hybridization states of coherent nucleic acid molecules or of polynucleotide sequences. Differently than in a real-time quantitative PCR method by fluorescence measurement in the UV regime, consequently detections can detect, corresponding to the method according to the invention, the presence of individual hybridization states, in particular, the presence of single-stranded and double-stranded polynucleotide sequences. This likewise quantitative detection, which is consequently decisive for the execution of a PCR amplification, allows conclusions to be made on the progress as well as the quality of the reaction steps taking place in the PCR solution and thus allows an increase of the efficiency of the PCR amplification process in general.

Another core concept of the method according to the invention lies furthermore in that the radiation source used for the spectroscopic real-time detection can also be used to bring about the reaction step of denaturation in the PCR amplification process through electromagnetic radiation. The irradiated frequency range is definitely suitable, namely, for melting the hydrogen bridge bonds between the two single strands in a double-stranded polynucleotide sequence, without having to add thermal energy directly to the entire system of the PCR solution. Consequently, in the reaction step of denaturation, a temperature increase of a PCR solution is not needed for generating the melting of the chemical bonds between individual strands of a polynucleotide sequence. Instead, through irradiation of electromagnetic radiation of a predetermined frequency in the gigahertz as well as terahertz regime into the PCR solution, the radiation source causes a melting of double-stranded polynucleotide sequences, without the PCR solution itself having to undergo a strong change in temperature. Such a method thus shortens the heating and cooling phases of the PCR solution needed for the repeating PCR reaction steps and allows a quicker time presentation of a desired quantity of polynucleotide sequences to be amplified.

In a first embodiment of the method according to the invention, it is provided that the initial substances necessary for the production of a PCR solution in a buffer solution comprise no chemical or physical detection markers, in particular no fluorescence markers. These include markers, which are added as initial substances that are not required for the production of a PCR solution described farther above, in order to be able to detect the presentation of polynucleotide sequences in a measurement method that is known from the prior art. The PCR solution provided for the method according to the invention consequently can be limited to the required initial substances, like those needed, for example, for a conventional PCR amplification without real-time detection. With this configuration, on the one hand, possible disruptive influences of a chemical as well as physical nature are reduced in the method of the PCR amplification process, and simultaneously the controllability of the reaction conditions is increased.

In another embodiment it can be provided that the intensity of the irradiated electromagnetic radiation in the gigahertz or terahertz regime is selected so that it penetrates a predetermined layer thickness of the PCR solution. The layer thickness can here relate either to the entire sample cross section, which is penetrated by the irradiated electromagnetic radiation or else even only to a sub-region of the sample cross section. The layer thickness is suitable to detect an extinction signal for the irradiation of a specified radiation intensity and presence of a number of polynucleotide sequences above a certain detection threshold. The extinction signal that results from an attenuation of the electromagnetic radiation intensity can result, for example, from absorption, scattering, diffraction, as well as reflection. If the penetrated layer thickness is constant, and also the irradiated electromagnetic radiation is invariable in its intensity, then a conclusion can be made on the quantity of presented polynucleotide sequences, for example, for a known extinction cross section of the polynucleotide sequences.

In a further embodiment of the method, it is provided that the frequency of the irradiated electromagnetic radiation lies in the gigahertz or terahertz regime between 100 GHz and 20 THz, in particular between 300 GHz and 10 THz. These frequency ranges cover the excitation frequencies, which can be easily detected in a spectroscopic manner for most polynucleotide sequences and thus allow excitation oscillations to be detected in a spectroscopic manner, in particular in a spectroscopic and quantitative manner.

In another advantageous embodiment of the invention, it is provided that the buffer solution of the PCR solution is selected with respect to a frequency of the irradiated electromagnetic radiation in the gigahertz or terahertz regime, so that the radiation intensity can be detected in an essentially non-attenuated way with the detector after transmission through a predetermined layer thickness of the PCR solution. The buffer solution itself is thus selected with respect to the electromagnetic radiation frequencies in use, so that the irradiated electromagnetic radiation of predetermined frequency ranges experiences a largest possible transmission. If these frequency ranges overlap with excited states to be detected in the polynucleotide sequences, then it is guaranteed that a reduction of the electromagnetic radiation intensity according to the interaction with the PCR solution is not based upon the extinction response of the buffer solution itself, but instead only on that of the initial substances and also with the presented molecules. Furthermore, if an extinction by byproducts of the PCR amplification process can be ruled out, then the attenuation of the electromagnetic radiation intensity in the regions of the excited states of the polynucleotide sequences is based essentially only on the interaction of the electromagnetic radiation with the polynucleotide sequences. The ratio of usable signal and undesired attenuation of the electromagnetic radiation is consequently increased and the quality of the likewise quantitative, spectroscopic real-time detection is improved.

In a further embodiment of the method, this distinguishes itself in that the PCR solution is deposited on a substrate, in particular in a predetermined absorption area of the substrate, wherein this area is essentially transparent for the irradiated electromagnetic radiation in the gigahertz or terahertz regime. Such an absorption area can be formed by a limited volume or instead only as a surface area for deposition of a PCR solution. The substrate itself can be a mineral substrate, for example glass, or instead a plastic substrate. If the substrate itself is essentially transparent for the irradiated electromagnetic radiation, it causes merely a small portion of the extinction signal detected in the spectroscopic real-time detection method. The detected extinction thus essentially is based upon the interaction of the electromagnetic radiation and the PCR solution or the substances contained therein, if the buffer solution of the PCR solution itself likewise causes no or only little attenuation of the irradiation intensity in the frequency range of the irradiated electromagnetic radiation. Furthermore, if it is a plastic, the substrate can be adapted to the usable frequency range of the irradiated electromagnetic radiation through the selection of a suitable plastic, in order to cause a lowest possible attenuation of the radiation intensity.

In another embodiment of the method, the substrate comprises at least one waveguide for electromagnetic radiation in the gigahertz or terahertz regime. This waveguide can be used, on the one hand, as radiation guiding means and also simultaneously as a substrate with a possible absorption area for the PCR solution. Accordingly, the irradiation of electromagnetic radiation into the PCR solution can be performed locally in an easily controllable manner. In addition, the scattering radiation or loss radiation, like that occurring in conventional collimation and focusing methods, can be reduced and the spectroscopic real-time detection can be improved. In an especially advantageous way, a waveguide can be used when the predetermined absorption area for the PCR solution is located in an area that is penetrated by the evanescent radiation field of the waveguide. A detection of the polynucleotide sequences consequently takes place with electromagnetic radiation, which is guided, on the one hand, by the waveguide, but on the other hand, communicates with areas of the surface outside of the waveguide.

Furthermore, in a further embodiment of the method for the spectroscopic real-time detection, a temperature-change device is provided by which the PCR solution can be heated and/or cooled to specified temperatures. Such a temperature-change device can heat or cool the PCR solution either directly or indirectly. A direct temperature increase can be based upon, for example, a direct radiation heating or a resistance heating. Cooling can be caused, for example, through suitably arranged Peltier elements or instead through a cooling medium, for example air that is connected to a heat exchanger. The temperature-change device here guarantees that a fastest and most precise change possible of the temperatures needed for the individual PCR reaction steps is enabled.

In a further embodiment of the method, it is provided that the temperature-change device heats and/or cools a heating medium, which heats and/or cools the PCR solution indirectly via the substrate. It is further to be taken into account that the heat capacity of the substrate itself is low and the thermal conductivity is as large as possible. According to this construction, the heat medium can interact over a surface area or instead this is penetrated in guide means provided for this purpose, for example in channels. By the heating or cooling of the substrate, for suitable contact of the PCR solution with the substrate, the temperature of the PCR solution is changed accordingly. Consequently, the temperature conditions required for the course of the PCR reaction steps can be guaranteed.

In one advantageous embodiment of the method, it is provided that electromagnetic radiation in the gigahertz or terahertz regime is irradiated from the radiation source into the PCR solution before and/or after a step of denaturation and/or primer hybridization and/or elongation.

Accordingly, the starting conditions and also reaction results after each of the reaction steps taking place in the PCR amplification process can be tested by a spectroscopic detection process. If it is discovered, for example, by the detection that the starting requirements for a subsequent PCR reaction step are unfavorable, corresponding changes of the reaction conditions, for example in the temperature selection, can be performed, in order to bring about an improved result. In one alternative construction, the electromagnetic radiation in the gigahertz or terahertz regime can also be irradiated in the reaction steps of denaturation, primer hybridization, or elongation taking place during the PCR amplification process, in order to obtain conclusions on the time bonding behavior of individual molecular building blocks.

In one especially advantageous embodiment of the method, the frequency of the electromagnetic radiation is selected, in order to excite defined vibration excitation modes of single-stranded and/or double-stranded polynucleotide sequences, in order to detect these modes, which are characteristic, in particular, for the presence of hybridization states of double-stranded polynucleotide sequences. Consequently, during the PCR amplification process, the presence of certain molecular structures of the polynucleotide sequences can be detected, by which the quality of the progress of the PCR amplification process can be checked and detected. Furthermore, after successful detection of the polynucleotide sequences, an interruption of the PCR amplification can also be initiated. In particular, by the detection of the shift of the excitation frequency of defined excitation modes, a conclusion on the structure of the polynucleotide sequences can also be obtained, in particular, on their hybridization state.

In another embodiment of the invention, during the PCR amplification process, prior detections are taken into consideration by the electromagnetic radiation in the gigahertz or terahertz regime as background for subsequent detections in the analysis of the detections. The prior detections can be removed from subsequent detections either in a direct, averaged, or filtered way for the background subtraction that is necessary in extinction measurements. This step of background subtraction, which is necessary for obtaining quantitative results, thus requires no additional prior knowledge on the actual extinction response of the PCR arrangement, but instead allows a good background determination in an approximately real-time way.

In another embodiment of the method for the spectroscopic real-time detection of polynucleotide sequences, electromagnetic radiation in the gigahertz or terahertz regime is irradiated from the radiation source before the execution of a PCR amplification process into a solution of initial substances to be used in the PCR amplification process, in order to determine chemical or physical properties of individual initial substances, in particular their quality, specificity, and their bonding behavior, by spectroscopic detections. Here, it is to be noted that typically before the execution of a real-time quantitative PCR process, most users of PCR amplification processes test the quality or specificity and the bonding behavior (quality of the polynucleotide sequences, primer bonding, primer specificity, amplification quantities) for reasons of cost in a PCR device, which does not maintain the possibility of a spectroscopic real-time detection under use of fluorescence measurements. According to one execution of the method according to this embodiment, this separate test can thus be eliminated and the physical and chemical properties of individual initial substances can be determined directly by a PCR arrangement according to the invention.

Furthermore, the primers typically used in PCR amplification processes are designed for use in conventional PCR arrangements without spectroscopic real-time detection and require, in the use in a PCR arrangement with spectroscopic real-time detection on the basis of the use of fluorescence markers, an innovative optimization of the reaction conditions for the PCR solution containing the primer with fluorescence markers. This renewed optimization is usually necessary, because the addition of fluorescence markers influences the melting points of the polynucleotide sequences and thus the primer bond. If the primer bond or primer specificity can be determined in advance in a PCR arrangement in which, at a later time point, the spectroscopic real-time detections of polynucleotide sequences can also be performed with gigahertz or terahertz radiation, expensive optimization requirements are thus eliminated.

Furthermore, it should also be noted that the reaction vessels used in fluorescence measurements in the UV regime can easily become a source of incorrect measurements due to contamination, as, for example, fingerprints, and incorrect absorption values can result. Electromagnetic radiation in the gigahertz or terahertz regime is definitely more insensitive with respect to such contamination of the reaction vessels and also the buffer-solution components, due to the greater wavelength. In addition, the use of corrupt or imprecisely set fluorescence dyes is also eliminated accordingly in the present method as a possible source of error for the spectroscopic detection.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments which are presently preferred. It should be understood, however, that the invention is not limited to the precise arrangements and instrumentalities shown. In the drawings:

FIG. 1 is a flowchart illustrating the time progress of individual steps in a method for the spectroscopic real-time detection of polynucleotide sequences in a PCR amplification process according to a first embodiment of the invention;

FIG. 2 is a schematic representation of the extinction response of a PCR solution in the course of a PCR amplification process at radiation frequencies that agree with excited states of predetermined polynucleotide sequences in a PCR solution;

FIG. 3 is a partial view of a schematic representation of a second embodiment of a PCR arrangement according to the invention;

FIG. 4 is a partial view of a schematic representation of a third embodiment of a PCR arrangement according to the invention;

FIG. 5 is a partial view of a schematic representation of a fourth embodiment of a PCR arrangement according to the invention;

FIG. 6 is a partial view of a schematic representation of a fifth embodiment of a PCR arrangement according to the invention;

FIG. 7 is a schematic representation of a sixth embodiment of a PCR arrangement according to the invention, which is based upon the partial view of the fourth embodiment shown in FIG. 5;

FIG. 8 is a schematic representation of the extinction response of a buffer solution used for the preparation of a PCR solution, as well as a polynucleotide sequence in the frequency regime of the gigahertz and/or terahertz regime.

### DETAILED DESCRIPTION OF THE INVENTION

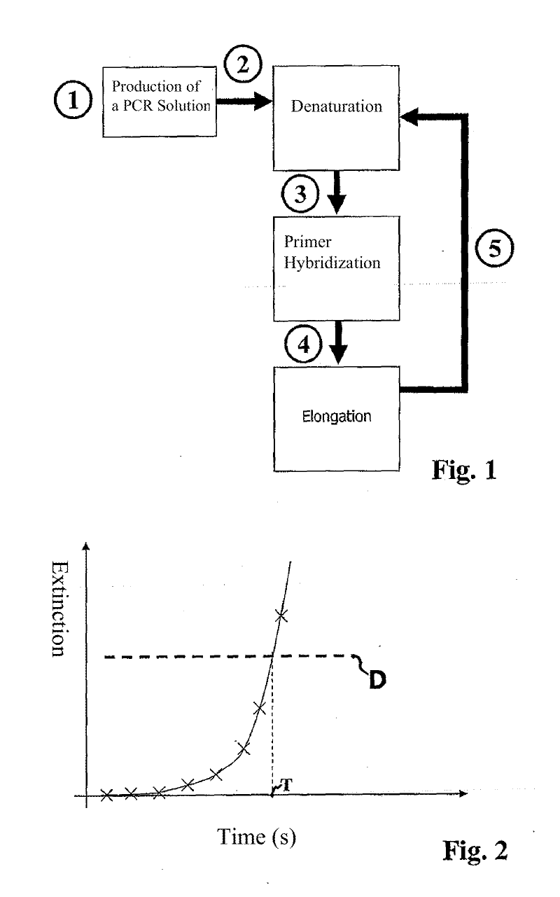

FIG. 1 shows a schematic flowchart of the typical progress of one embodiment of the method according to the invention for the spectroscopic real-time detection of polynucleotide sequences in a PCR amplification process. In such a method, it requires initially the preparation of a buffer solution in which the initial substances necessary for the production of a PCR solution are absorbed or dissolved. Required initial substances are the polynucleotide sequence to be replicated, suitable primer, a polymerase, as well as deoxynucleoside triphosphates for the synthesis of polynucleotide sequences. Even before the preparation of the PCR solution, the quality, specificity and the formation behavior of individual initial substances can be determined in a spectroscopic real-time detection process. This detection takes place at a time point 1 illustrated in FIG. 1.

After preparation of the PCR solution, new, additional spectroscopic real-time detections of the PCR solution can be performed (time point 2). An amplification of the polynucleotide sequences then takes place only through the repetition of the PCR reaction steps of denaturation, primer hybridization, and elongation, wherein after each successful PCR reaction step, spectroscopic real-time detections can be performed (time points 3, 4, and 5). Here, preferred excited states of the single-stranded polynucleotide sequences are measured after the PCR reaction step of denaturation (time point 3), wherein excited states of double-stranded polynucleotide sequences are measured in a suitable way after the PCR reaction step of elongation (time point 5).

By the method according to this embodiment, spectroscopic real-time detections can be performed during the entire progress of the PCR amplification process and the time course of the polynucleotide sequences occurring in the PCR amplification process is also characterized in a quantitative manner. Furthermore, it is naturally also possible to still perform spectroscopic detection measurement after completion of the PCR amplification process.

According to one aspect of the present method according to the invention, if the PCR reaction step of denaturation is to be caused through irradiation of electromagnetic radiation in the terahertz regime, then this method would perform an irradiation of corresponding electromagnetic radiation at the time point of the completion of the denaturation.

FIG. 2 shows a schematic representation of the extinction course in a PCR solution, which can be determined according to one embodiment of the present invention with electromagnetic radiation in the gigahertz or terahertz regime. The frequency of the irradiated electromagnetic radiation is here selected so that this agrees with the frequency of predetermined excited states of individual polynucleotide sequences and is attenuated by these after corresponding irradiation into the PCR solution. Because the number of polynucleotide sequences to be replicated typically in a PCR amplification increases exponentially in time, the measurement points of spectroscopic real-time detections for extinction lie on a curve with an exponential course. In the present case, the individual detections of the extinction of the PCR solution are illustrated by crosses. The time course is here determined according to physical, as well as chemical, parameters that can influence the efficiency or rate of the PCR amplification. Then, the quantity of specific polynucleotide sequences can be determined from the extinction measurements by computational methods.

In the practical execution of spectroscopic real-time detections of polynucleotide sequences during the PCR amplification process, one finds that only those detections above a predetermined detection threshold D₁ deliver unambiguous detection results that can also be used for further evaluation. Detection values below this detection threshold D₁ are merely of a theoretical nature, because the signal-to-noise ratios do not allow clear extinction determinations. The earliest time point T₁ for an unambiguous spectroscopic real-time detection consequently occurs when the measured extinction lies above the detection threshold D₁. Through a corresponding frequency selection of the irradiated electromagnetic radiation, it can be achieved that a detection threshold D₂ is as low as possible and lies below the detection threshold D₁ for electromagnetic radiation of other frequencies. Consequently, fewer PCR reaction steps must be performed than in conventional real-time quantitative PCR methods. Furthermore, the earliest time point T₂ is also as small as possible, in particular, shorter than T₁ for electromagnetic radiation of the other frequencies.

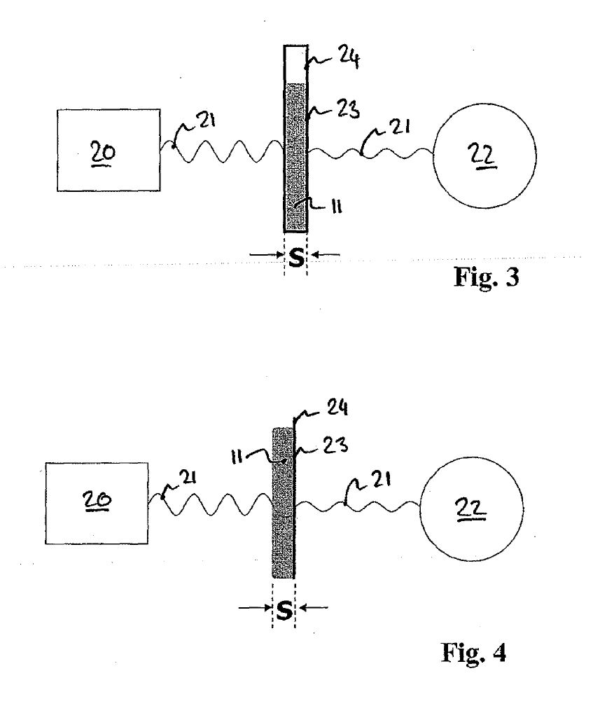

FIG. 3 shows a partial view of a schematic diagram of an embodiment of a PCR arrangement according to the invention for the spectroscopic, real-time detection of polynucleotide sequences in a PCR amplification process. According to this embodiment, a radiation source 20 emits electromagnetic radiation in the gigahertz and/or terahertz regime in a PCR solution 11. The PCR solution 11 is located in the absorption area 23 of a substrate 24 that defines, through its form, the layer thickness S of the PCR solution through which the electromagnetic radiation 21 radiates. In the present case, such an absorption area 23 is formed by an at least partially limited volume, like a container. The frequency of the radiation is set here such that it agrees with the frequency of predetermined excited states of polynucleotide sequences located in the PCR solution 11. After transmission of the irradiated electromagnetic radiation 21 through the cross section of the absorption area 23, the correspondingly attenuated intensity of the electromagnetic radiation 21 is detected by a detector 22.

In the present case, it was omitted to specify the additional optical elements typical in the optical configuration, such as focusing elements, collimation elements, beam-guiding elements, and filters. The additional provision of such conventional, optical elements for beam conditioning presents itself as obvious for someone skilled in the art.

FIG. 4. shows another partial view of an embodiment of a PCR arrangement for the spectroscopic real-time detection of polynucleotide sequences in a PCR amplification process. In contrast to the absorption area 23 of the first embodiment according to FIG. 3, the second embodiment according to FIG. 4 provides no double-walled absorption area 23, but instead only a single-walled area on which the PCR solution 11 is absorbed. Here, it can involve, for example, a film deposit of a PCR solution on the absorption area 23 of the substrate. Furthermore, the arrangement of the PCR solution 11 can also be oriented on the absorption area 23 of the substrate 24 in the field of earth's gravitational force, so that during the spectroscopic real-time detection, a constant layer thickness S can be guaranteed. In addition, the absorption area 23 of the substrate 24 can still contain structural arrangements that increase the adhesion between the PCR solution 11 and the absorption area 23 of the substrate 24. Such arrangements are, for example, fine surface structures that support adhesion of the PCR solution 11 on the substrate 24.

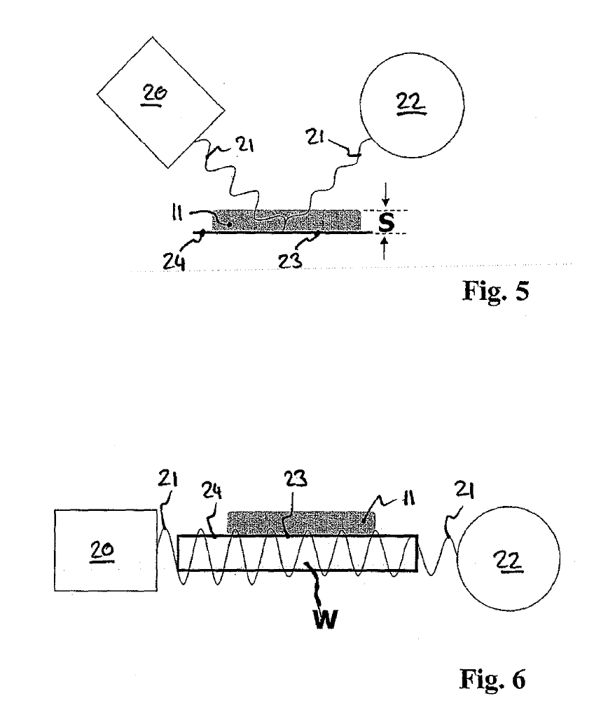

FIG. 5 shows a partial view of a fourth embodiment of the PCR arrangement according to the invention for the spectroscopic real-time detection of polynucleotide sequences in a PCR amplification. Here, the direction of the electromagnetic radiation 21 emitted from the radiation source 20 and the direction of the radiation detected by the detector 22 are not arranged in extension to each other. Instead, the electromagnetic radiation 21 is deflected by the PCR solution 11 and/or the substrate of the absorption area 23, such that no straight-line beam path is realized. Such a beam path is advantageous, for example, for scattering measurements. Such a relative arrangement of radiation source 20 and detector 22 can also contribute to an improved spatial arrangement of various components, which contributes to the reduction of the overall size of the PCR arrangement according to this embodiment.

In comparison with the embodiments of the free beam path of the electromagnetic radiation 21 in the diagrams according to FIG. 3 to FIG. 5, in the partial view of the embodiment according to FIG. 6, the electromagnetic radiation 21 is guided in a waveguide W suitable for the frequency range of the radiation. Here, the electromagnetic radiation 21 can be coupled into the waveguide W directly at the radiation source 20 or, however, only after suitable conditioning. Accordingly, the waveguide W can be in connection directly with the detector 22, or first the radiation can be conditioned for the detection by the detector 22. The waveguide W is constructed to discharge electromagnetic radiation 21 in the absorption area 23 as an evanescent radiation field, which can consequently generate an extinction of the electromagnetic radiation 21 due to the interaction with a PCR solution deposited on the waveguide W on the outside. The embodiments of the waveguide W can comprise insulated waveguide structures or, however, waveguide sections also integrated in additional absorption devices. In one preferred embodiment, the waveguide W is integrated into a chip construction.

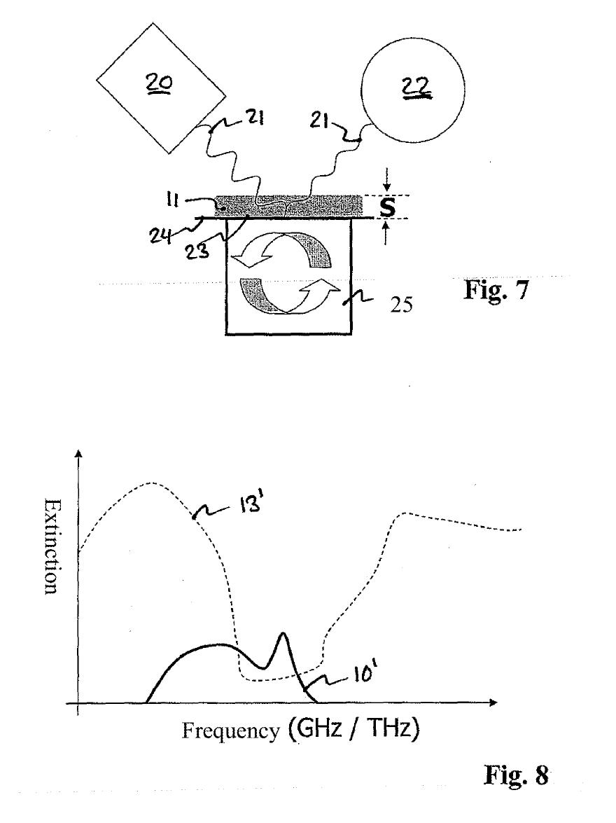

FIG. 7 shows an embodiment of a PCR arrangement based upon the partial view of the fourth embodiment in FIG. 5 for the spectroscopic real-time detection of polynucleotide sequences in a PCR amplification process that presents, as another element, the temperature-change device 25 interacting with the substrate 24 of the absorption area 23. The temperature-change device 25 is constructed to heat and/or to cool the substrate 24 on the side lying opposite the side of the absorption area 23 for absorption of the PCR solution 11. By changing the temperature of the substrate 24, through corresponding heat conduction, a temperature change of the PCR solution 11 is realized. If the heat resistance of the substrate 24 is low and also the heating and/or cooling performance of the temperature-change device 25 is large in comparison with the heat capacity of the deposited PCR solution 11, a relatively quick temperature change of the PCR solution 11 is realized, and a rapid progression of the repeating PCR reaction steps can be guaranteed. For improved heat conduction between the temperature-change device 25 and also the substrate 24, there can be means that support heat conduction. In one embodiment of the PCR arrangement according to the invention, the temperature-change device 25 can heat and/or cool an air flow, which is guided directly onto the substrate 24 and changes the temperature of the PCR solution 11 accordingly after corresponding heat conduction through the substrate 24.

FIG. 8 shows the extinction response 13′ of the buffer solution needed for the preparation of the PCR solution 11, as well as the extinction response 10′ of predetermined excited states of the polynucleotide sequence to be detected in a frequency range of the gigahertz or terahertz regime. Due to its diffraction response, the extinction of the buffer solution frequency range in a detectable manner. Many typical laboratory buffer solutions exhibit such an extinction response. For the lowest possible noise detection of an excited state of a polynucleotide sequence, it can be necessary to select a buffer solution, which has, in the frequency range of the designated excited state of the polynucleotide sequence a minimum of its extinction response. Accordingly, the extinction of the electromagnetic radiation 21 irradiated into the PCR solution 11 through the buffer is realized with only little disruption through the buffer solution. Through corresponding selection or setting of the buffer solution to the polynucleotide sequence to be detected and its excitation frequency, an advantageous signal-to-noise ratio can be achieved.

At this point it should be noted that all of the parts described above are claimed as essential to the invention viewed alone and in any combination, in particular the details illustrated in the drawings. Changes to these parts are familiar to someone skilled in the art.

It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

I/We claim:

1-17. (canceled)

18. A method for spectroscopic real-time detection of nucleic acid molecules in a PCR amplification process, the method comprising:
preparing initial substances necessary for production of a PCR solution in a buffer solution for the PCR amplification process;
repeating PCR reaction steps of denaturation, primer hybridization, and elongation during the PCR amplification process;
irradiating electromagnetic radiation from a radiation source in a gigahertz or terahertz regime into the PCR solution at defined time points during the PCR amplification process; and
detecting at least a presence or absence of a polynucleotide sequence by a detector in a real-time detection.

19. The method according to claim 18, wherein the nucleic acid molecules comprise polynucleotide sequences.

20. The method according to claim 18, wherein the initial substances necessary for production of the PCR solution in a buffer solution comprise no chemical or physical detection markers, in particular no fluorescence markers.

21. The method according to claim 18, wherein an intensity of the irradiated electromagnetic radiation is selected in the gigahertz or terahertz regime, such that it penetrates a predetermined layer thickness (S) of the PCR solution.

22. The method according to claim 18, wherein a frequency of the irradiated electromagnetic radiation in the gigahertz or terahertz regime lies between 100 GHz and 20 THz, optionally between 300 GHz and 10 THz.

23. The method according to claim 18, wherein the buffer solution of the PCR solution is selected in the gigahertz or terahertz regime with respect to a frequency of the irradiated electromagnetic radiation, such that a radiation intensity can be detected essentially non-attenuated with the detector after transmission through a predetermined layer thickness (S) of the PCR solution.

24. The method according to claim 18, wherein the PCR solution is deposited on a substrate in a predetermined absorption area.

25. The method according to claim 24, wherein the predetermined absorption area of the substrate is essentially transparent for the irradiated electromagnetic radiation in the gigahertz or terahertz regime.

26. The method according to claim 24, wherein the substrate comprises at least one waveguide (W) for electromagnetic radiation in the gigahertz or terahertz regime.

27. The method according to claim 18, further comprising heating and/or cooling the PCR solution to a predetermined temperature by a temperature-change device.

28. The method according to claim 27, wherein the temperature-change device heats and/or cools a thermal medium, which directly or indirectly heats and/or cools the PCR solution by the substrate.

29. The method according to claim 18, wherein the electromagnetic radiation in the gigahertz or terahertz regime is irradiated from the radiation source into the PCR solution before and/or after a step of denaturation and/or primer hybridization and/or elongation.

30. The method according to claim 18, wherein a frequency of the electromagnetic radiation is selected to excite defined vibration excitation modes of single-stranded and/or double-stranded polynucleotide sequences, such that these modes may be detected.

31. The method according to claim 30, wherein the modes are characteristic for the presence of hybridization states of double-stranded polynucleotide sequences.

32. The method according to claim 18, wherein during the PCR amplification process, prior detections by the electromagnetic radiation in the gigahertz or terahertz regime are taken into consideration as background for subsequent detections in an analysis of the detections.

33. The method according to claim 18, wherein electromagnetic radiation in the gigahertz or terahertz regime is irradiated from the radiation source before execution of a PCR amplification process into a solution of initial substances to be used in the PCR amplification process, in order to determine chemical or physical properties of individual initial substances by spectroscopic detection.

34. The method according to claim 33, wherein the chemical or physical properties of individual initial substances determined by spectroscopic detection are selected from their quality, specificity, and bonding behavior.

35. A PCR arrangement for spectroscopic real-time detection of polynucleotide sequences in a PCR amplification process, the arrangement comprising:
a substrate having an absorption area for a PCR solution comprising necessary initial substances and a buffer solution;
a temperature-change device for heating and/or cooling the PCR solution to specified temperatures to allow repeating PCR reaction steps of denaturation, primer hybridization, or elongation;
a radiation source for irradiating electromagnetic radiation in a gigahertz or terahertz regime into the PCR solution; and
a detector for detecting, in a spectroscopic manner, radiation transmitted through the PCR solution.

36. A method for spectroscopic real-time detection of nucleic acid molecules in a PCR amplification process, the method comprising:
preparing initial substances necessary for production of a PCR solution in a buffer solution for the PCR amplification process;
repeating PCR reaction steps of denaturation, primer hybridization, and elongation during the PCR amplification process;
irradiating electromagnetic radiation from a radiation source in a gigahertz or terahertz regime into the PCR solution at defined time points during the PCR amplification process; and
detecting at least a presence or absence of a polynucleotide sequence in a spectroscopic real-time detection process by a detector,
wherein the reaction step of denaturation is caused by terahertz radiation from the radiation source.

37. The method according to claim 36, wherein the nucleic acid molecules comprise polynucleotide sequences.
